(19) United States
(12) Patent Application Publication
(10) Pub. No.: US 2019/0241344 A1
(43) Pub. Date: Aug. 8, 2019

(54) LEAVEN

(71) Applicant: Pascal PHILIBERT, Replonges (FR)

(72) Inventors: Pascal PHILIBERT, Replonges (FR); Annabelle VERA, Craponne (FR)

(21) Appl. No.: 16/266,272

(22) Filed: Feb. 4, 2019

(30) Foreign Application Priority Data
Feb. 2, 2018 (FR) 18 70117

(51) Int. Cl. B65D 81/20; B65D 81/24; C12N 1/04

(52) U.S. Cl. CPC B65D 81/2069; B65D 2565/387; C12N 1/04; B65D 81/24

(57) ABSTRACT

This product based on leaven comprises a package containing living leaven closed with a gas tight seal. The leaven is in a nitrogen atmosphere.

### The present invention relates to the packaging of leaven or the like to ensure good preservation conditions and Prolong the life of the product.

“Leaven and the like” means any fermented product based on cereals (soft wheat, hard wheat, rye for example), pseudo-cereals (buckwheat, quinoa for example); or legumes, and all their forms (dry whole grain, germinated seeds, flour, flour mixtures, semolina and all other fractions (brans, wheat germ for example).

Leaven is a traditional fermented product, used since ancient times, to make bread and all kinds of leavened doughs, containing live microflora consisting mainly of lactic bacteria and yeasts. It can be produced by the spontaneous fermentation of microorganisms naturally occurring in the fermentation substrate, generally flour, or based on seeding with selected microorganisms known as starters.

The fermentation of microorganisms in leaven, in particular lactic bacteria, during bread making is essential and gives bread made with leaven all its aromatic richness and typical tangy taste. The metabolically active microorganisms release organic acids, numerous aromatic compounds and precursors of aromas, and also other compounds of interest such as exopolysaccharides and fungicides and begin the breakdown of the constituents of the dough during bread making. Thus, living leaven affects all the organoleptic attributes of the bread (more texture of the crumb, irregular honeycombing, a thicker and crisper crust), improves its keeping, slows down staling and the growth of moulds, and improves the digestibility of the bread, by encouraging in particular the bioavailability of minerals and reducing its glycemic index.

Living leaven differs from devitalised leaven firstly in its microbiological composition, meaning that devitalised leaven no longer contains living microorganisms and has lost its fermenting action. Living leaven is generally stabilised by the cold, refrigerated and does not stay fresh for so long, while devitalised leaven, in which the microflora has been destroyed by various processes, such as high temperature dehydration, pasteurisation or even by osmotic pressure, generally stays fresh for a long time at unregulated ambient temperature. However, devitalised leaven no longer has fermentation action and is only used aromatise the dough.

The invention concerns all types of leaven or similar which contain a percentage of dry material (MS) of less than 90% by weight, in particular less than 75% or 70° and that are active/living which may undergo changes in their organoleptic properties associated with oxygen, and nutritional and microbian properties while kept in a below zero or unregulated ambient temperature. It is a matter of oxidation phenomena, enzymatic or non-enzymatic browning, enzymatic or even microbian degradation.

These changes associated with the oxygen in the air lead to an organoleptic reduction in quality (a change in the colour or aroma, for example), nutritional quality (oxidation of the unsaturated fatty acids, vitamins for example) and microbial growth of the leaven or similar (growth of undesirable aerobic flora, for example moulds, or reduction in technological flora, in particular lactic anaerobic bacteria).

WO 02/02249 (Liquid Air) concerns the packing of perishable products, which are particularly sensitive to the growth of microorganisms intended to prolong their storage life while ensuring their microbiological safety.

It Also Specifies that:

“Microbiological safety” means conditions of storage that prevent, or at least reduce, the growth of microorganisms to an acceptable degree, that is to say non-toxic for the consumer and satisfying current regulations for the products in question.

The term “microorganisms” includes not only pathogenic microorganisms, but also non-pathogenic microorganisms that can change the properties and/or activity of the product, in particular those that can give it an unpleasant appearance or odour or alter or degrade the nutritional, chemical and/or pharmaceutical properties of the products.

WO 02/02409 (Liquid Air) claims packing with a protective gas formed of hydrogen and maybe a packaging gas, which may be inert like nitrogen or argon, or even a gas that may have a protective interaction such as carbon dioxide or nitrous oxide.

The purpose of Liquid Air is to avoid and reduce the growth of microorganisms in foodstuffs. But, leaven is a living product in which it is desirable to preserve the action of the characteristic microorganisms it contains; lactic bacteria and yeasts. Preservation of the microorganisms in leaven means keeping the microorganisms in a viable (living) and cultivable state, that is to say that they are capable of continuing metabolic activity under more favourable conditions. Leaven is a naturally stable product in respect of undesirable flora, since it is an acid product.

The tests in example 3 of preservation of leaven on the basis of the present invention showed that only a protective atmosphere of essentially 100% nitrogen N2, which is an inert gas, was able to stabilise the level of lactic bacteria in the leaven. An atmosphere containing hydrogen, in particular 4.5% H2/95.5% N2 was shown to be harmful after 6 weeks of preservation, like air. Concerning the level of lactic bacteria, a deviation of >0.5 log(ufc)/g was considered a “true” difference. These tests showed that the difference between packing in 100% N2 and 4.5% H2/95.5% N2 was 1.77 and 1.68 log(ufc)g for 6 and 8 weeks of storage respectively for leaven, or 47.75 times more living bacteria present after 8 weeks of storage for leaven in N2. The invention also enables the content of fatty acids, in particular unsaturated fatty acids in leaven to be maintained (stabilised).

The subject of the invention is a product consisting of leaven or the like having a percentage of dry material by weight of less than 90%, living, characterised in that the product is contained in an atmosphere of nitrogen of purity greater than 97%, preferably between 99 and 100%, by volume packed in a gas barrier material and closed with a gas tight seal.

The leaven or the like is packed in a manner that limits contact with oxygen. The leaven is packed in a manner that has a residual level of O₂ in the atmosphere inside the package of <5%, preferably <2%. For that, the leaven is preferably packed in a modified protective atmosphere in a package made from a gas barrier material. The package is closed with a gas tight seal.

The protective atmosphere must be non-oxidising. The ambient air in a packaging container is replaced with nitrogen. The nitrogen (N₂) should have a purity of >97%, in particular from 99 to 100% by volume.

The material of the packaging container may be of any type (composition), flexible or rigid, and must form an effective barrier to gases, mainly oxygen (O₂), carbon dioxide (CO₂), nitrogen (N₂) and water vapour. More particularly for oxygen, the material of the packaging container must have a permeability coefficient to O₂ of <30 cm³/m²*/24 h.bar at 23° C., preferably <10 cm³/m²/24 h.bar at 23° C. The material should preferably be a multilayer structure forming an effective barrier to gases. For example, it may consist, among other things of PVDC (Polyvinylidene chloride), OPA (oriented polyamide), EVOH (polyethylene vinyl alcohol), aluminium, silica type SiOx and/or glass or other material. The packaging material should preferably be anti-UV.

The package can be of any shape (covered tray or pack, for example).

The packaging can be made by any machine that can produce a package under vacuum or with a protective atmosphere, preferably using gas scanning techniques or by compensated vacuum with gas injection. The space occupied by the gas (known as the head space) must be >10% of the total volume of the package and the atmosphere must have a residual O₂ level of <5%.

The subject of the invention is also a process in which the leaven or the like, is placed, living, in a container made from a gas barrier material closed with a gas tight seal.

The Following Methods are Used:

Method of analysing gaseous composition: The gaseous composition in O2 and CO2 of the head space is measured by the use of an O2/CO2 analyser 30 Oxybaby (manufacturer's trade name) model 6.0 (WIT). For each analysis point, the method is destructive (puncturing the package and analysing the gas in the head space in the pack). The pack is then opened for the following analyses. The analyser measures the levels of O2 and CO2 directly with a probe then calculates the percentage of gases other than O2 and CO2 contained in the gaseous atmosphere by difference, the sum of all the gases being 100%. In the tests in the example, the other gases consist mainly of N2 in example 3A, N2 only in example 3B and H2+N2 in example 3C.

Method of analysis of lactic bacteria: The level of lactic bacteria was determined in accordance with reference standard NF ISO 15214 (classification index V 08-030), the reference method, in the following manner: The surface was seeded on agar MRS (Biokar (manufacturer's trade name) previously poured into petri dishes with a determined quantity of mother suspension and/ or decimal dilutions of the sample. The enumeration was done after anaerobic incubation of the dishes at 30° C. for 72 to 120 hours. The result was expressed as the log to base ten of the units forming colonies per gram of leaven (log(ufc)/g).

Method of analysis of fatty acids: the total content of fatty materials and the composition in fatty acids were measured respectively by gravimetry (Soxhlet) and by the GC/FID technique. The fatty material was hydrolysed and extracted with sulphuric acid and cyclohexane using microwave techniques. The fatty material was determined by gravimetry after evaporation of the cyclohexane.

To profile the fatty acids, the fatty material was extracted, then prepared by methylation and analysed by chromatography in the gaseous phase (CPG) with direct sampling. The results were expressed as a percentage of the total sum of the fatty acids comprising the leaven.

Only fatty acids representing more than 0.05% of the total fatty acids were quantified.

The dry material (MS) was measured by the infrared method with a Radwag (manufacturer's trade name) halogen desiccator, mode MAC 50/1. Three measurements were made for 2.0±0.1 g of product, then averaged. The analysis program applied a temperature of 130° C. until the reduction in weight was ≤1 mg in 25 s. The values were expressed as a percentage of MS contained in the leaven or the like.

The colour of the leaven was monitored by instrumental analysis with a MINOLTA CR410 colorimeter for the chromatic space L*a*b*, for which the colorimetric coordinates measured were:
“L*” : black/white level (on a scale of 0=pure black to 100=pure white);
“a*” : green/red level (on a scale of −60=pure green to +60 =pure red);
“b*” : level blue/yellow (on a scale of −60=absolute blue to +60=absolute yellow).

### EXAMPLE NO. 1

500 g of wheat germ leaven whose MS was 32.5% (fresh living leaven) were packaged in a PET pack coated with PVDC-PE with a permeability coefficient to O₂ of <10 cm³/m²/24 h.bar, in a protective atmosphere consisting of 100% nitrogen (MESSER (manufacturer's trade name)) for food use in accordance with CE rule no. 1333/2008. The packaging was done using a compensated vacuum jar CON (manufacturer's trade name) series BORA 550 Dual Gas according to the following the parameters: 8 seconds pulling vacuum, 8 seconds injection of gas at a pressure of 3 bar, 5 seconds heat sealing. The volume occupied by the protective atmosphere (head space) represented 40% of the total volume in the pack.

The packed leaven was then kept in a classical cold chamber at a temperature of 4-6° C. in a normal air atmosphere (consisting of about 20 to 21% O₂).

### EXAMPLE NO. 2: COMPARATIVE

5kg of wheat germ leaven with a MS of <32.5% % (fresh living leaven) was packed in a polypropylene bucket with a permeability coefficient to O₂ of 40 cm³/m²/24 h.bar. The packaging was carried out under normal air conditions, no protective atmosphere, nor placing under vacuum was applied. The volume of the head space in the bucket was 20% of the total volume of the bucket. The leaven in the bucket was then kept in a classical cold chamber at a temperature of 4-6° C. in a normal air atmosphere (consisting of about 20 to 21% O2).

The dry material (MS) was measured by the infrared method with a Radwag (manufacturer's trade name) halogen dessiccator model MAC 50/1. Three measurements were made for 2.0±0.1 g of the product, then averaged. The analysis program applied a temperature of 130° C. until the weight changed by ≤1 mg in 25 s. The values were expressed as a percentage of MS contained in the leaven or the like.

The gaseous composition in O₂ and CO₂ of the head space was measured with an Oxybaby (manufacturer's trade name) O₂/CO₂ model 6.0 analyser (WITT). For each measurement point, two packs were analysed by the destructive method (puncturing the package and analysing the gas in the head space of the pack).

For example 1: the colour was measured for the leaven previously homogenised in the pack (by manual pressure on the pack).

For example 2: a sample was taken of the leaven contained in the PP bucket. 50 g of the top layer of the leaven in contact with the head space (about 2 cm in height) were taken and 50 g of the lower layer then they were mixed. The colour measurement was made on this mixture.

Results in Table 1

| | | | Example 1 | Example 2 |
|---|---|---|---|---|
| Residual level of | 3 | days | 1.2 | — |
| O₂ in the head | 0.5 | months | 0.1 | — |
| space (in %) | 1 | months | 0 | — |
| | 1.5 | Months | 0.4 | — |
| Colour parameter L* | 3 | days | 67.16 | 65.59 |
| | 0.5 | months | 67.93 | 43.46 |
| | 1 | months | 68.08 | 46.44 |
| | 1.5 | months | 68.11 | 44.91 |
| Colour parameter a* | 3 | days | 4.12 | 2.72 |
| | 0.5 | months | 4.08 | −0.88 |
| | 1 | months | 4.36 | −1.23 |
| | 1.5 | months | 4.32 | −0.84 |
| Colour parameter b* | 3 | days | 20.83 | 19.01 |
| | 0.5 | months | 20.32 | −2.12 |
| | 1 | months | 20.67 | −0.5 |
| | 1.5 | months | 20.92 | −0.87 |

The results obtained show that in example 1 the residual level of oxygen inside the package was <2%, as recommended in the invention, and that that is sufficient to maintain a stable colour throughout the preservation of the leaven. There were only small variations in the colorimetric parameters L*a*b*, as the leaven retained its original light brown colour.

On the other hand, in example 2 the atmosphere in the package was not changed, the head space therefore consisted of normal air during packaging (the air consisting of 20-21% O2) and the PP bucket was not an adequate barrier to oxygen. This was translated into a significant change in the colouring (brown/black) of the leaven during preservation), mainly for the colorimetric parameter L* whose measurement exceeded 65.59 (fairly light leaven) at 44.91 after 1.5 months storage (leaven very dark grey/black). The leaven also changed towards green and blue for the parameters a* and b.

Examples 3A and 3C (Comparative) and 3B According to the Invention:

Tests Conducted in Air or Hydrogen Compared with Nitrogen:

500 g of wheat germ leaven with an MS of 32.5% by weight (fresh living leaven) were packed in an OPP/PE-EVOH-PE pack whose permeability coefficient to O2 is 2 cm3/m2/24 h.bar in an unmodified atmosphere consisting of air (example 3A) or in a modified atmosphere consisting of 100% nitrogen (MESSER (manufacturer's trade name)) (example 3B) or consisting of 4.5% H2/95.5% N2 (LINDE (manufacturer's trade name) (example 3C). Packing was carried out with a compensated CCM (manufacturer's trade name) vacuum jar series BORA 550 Dual Gas according to the following parameters: 8 seconds pulling vacuum, 8 seconds injection of gas at a pressure of 3 bar, then 3 or 4 seconds heat sealing.

The volume occupied by the protective atmosphere (head space) represented 40 to 50% of the total volume in the pack.

The packed leaven was then kept in a classical cold chamber at a temperature of 4-6° C. in an atmosphere of normal air (containing about 20 to 21% O2).

The leaven was monitored for 8 weeks, in particular the gaseous composition of the head space in the packs, the colour of the leaven, its composition in lactic bacteria and its composition in fatty acids.

Changes in Gaseous Composition

| | Storage | Level | Level | Other gases = |
|---|---|---|---|---|
| | time in | of O2 | of CO2 | level of N2 |
| | weeks | in % | in % | or N2 + H2 |
| In air | 0 | 21.13 | 0.4 | 78.5 |
| (3A) | 0.5 | 19.1 | 5.4 | 75.5 |
| | 1 | 18.4 | 7.3 | 74.3 |
| | 2 | 17 | 8.2 | 74.8 |
| | 3 | 16.1 | 11.1 | 72.8 |
| | 4 | 20.3 | 2.4 | 77.3 |
| | 6 | 9.1 | 13.5 | 77.4 |
| | 8 | 12.5 | 5.2 | 82.3 |
| In 100% N2 | 0 | 0.33 | 0.5 | 99.2 |
| (3B) | 0.5 | 2.3 | 2.8 | 94.9 |
| | 1 | 0.5 | 7.1 | 92.4 |
| | 2 | 1.4 | 6.3 | 92.3 |
| | 3 | 0.4 | 13.3 | 86.3 |
| | 4 | 1 | 9.7 | 89.3 |
| | 6 | 0.6 | 9.6 | 89.8 |
| | 8 | 0.6 | 7.4 | 92 |
| In 4.5% H2 + | 0 | 0.2 | 0.2 | 99.6 |
| 95.5% N2 | 0.5 | 0 | 4.2 | 95.8 |
| (3C) | 1 | 1.4 | 7.9 | 90.7 |
| | 2 | 0.1 | 5.2 | 94.7 |
| | 3 | 0 | 9.2 | 90.8 |
| | 4 | 0.5 | 10.6 | 88.9 |
| | 6 | 1.2 | 10.4 | 88.4 |
| | 8 | 0.5 | 7.4 | 92.1 |

The results obtained showed that the levels of residual oxygen inside the packs in a modified protective atmosphere of 100 % N2 or 4.5% H2/95.5% N2 complied with the recommendations of the invention. These are <5% residual O2 and predominantly <2% residual O2, while the packs packed in air showed residual levels of O2 in the head space in the pack of >9%, mostly >15% O2.

Leaven being a living product, the microorganisms, lactic bacteria and yeasts it contains have their metabolic activity greatly slowed down but not totally deactivated.

This may explain the production of CO2 in the packs in a protective atmosphere due to the fermentation metabolism of the microflora. In air, part of the oxygen was also consumed by the microorganisms.

Change in Colour

| | Storage | | | |
|---|---|---|---|---|
| | time in | | | |
| | weeks | L* | a* | b* |
| In air | 0.5 | 61.65 | 2.13 | 16.72 |
| | 1 | 57.29 | −0.14 | 11.31 |
| | 2 | 59.76 | 0.88 | 14.52 |
| | 3 | 56.21 | −0.21 | 10.13 |
| | 4 | 56.45 | −0.27 | 20.42 |
| | 6 | 56.73 | 0.09 | 11.28 |
| | 8 | 54.25 | 0.08 | 9.15 |
| In 100% | 0.5 | 62.43 | 2.86 | 17.89 |
| N2 | 1 | 62.55 | 3.16 | 18.38 |
| | 2 | 63.85 | 3.86 | 19.72 |
| | 3 | 63.29 | 3.61 | 18.89 |
| | 4 | 63.24 | 3.42 | 18.75 |
| | 6 | 62.58 | 3.39 | 18.74 |
| | 8 | 62.78 | 3.89 | 18.86 |
| In 4.5% | 0.5 | 63.4 | 4.22 | 20.77 |
| H2 + | 1 | 63.63 | 4.04 | 19.92 |
| 95.5% N2 | 2 | 64.23 | 4.22 | 20.25 |
| | 3 | 64.03 | 4.19 | 19.85 |
| | 4 | 64.07 | 4.19 | 20.17 |
| | 6 | 63.96 | 4.26 | 20.22 |
| | 8 | 62.26 | 3.84 | 18.73 |

The modified 10.0% N2 and 4.5% H2/95.5% N2 atmospheres had an equivalent effect on the colour of the leaven. Both of them permitted the colour properties of the leaven to be retained, thus preventing oxidation of the leaven, while there was a significant change in colour in air. The diagram above shows the effect on colour parameter L* in the L*a*b * system in which the value reduces rapidly as shown by colouration of the leaven which tends significantly to black, making the leaven unfit for sale, while in the 100% N2 and 4.5% H2/95.5% N2 modified atmospheres the parameter L* remained stable, like the ether parameters a* and b*.

Change in the Lactic Microflora

| | Level of lactic bacteria in | | |
|---|---|---|---|
| | log (ufc)/g | | |
| | | | In 4.5% |
| Number | | In 100% | H2 + |
| of weeks | In air | N2 | 95.5% N2 |
| 0.5 | 7.11 | 7.65 | 7.58 |
| 1 | 7.38 | 7.48 | 7.62 |
| 2 | 7.32 | 7.00 | 7.30 |
| 3 | 7.52 | 7.40 | 7.30 |
| 4 | 5.30 | 7.18 | 6.78 |
| 6 | 4.04 | 6.85 | 5.08 |
| 8 | 4.85 | 6.96 | 5.28 |

Change in the Fatty Acids Content

| | Storage | | Profile of fatty acids in % | | | Difference from t0 | | |
|---|---|---|---|---|---|---|---|---|
| | time in | Total MG | AG | AG mono- | AG poly- | AG | AG mono- | AG poly- |
| | weeks | g/100 g | saturated | unsaturated | unsaturated | saturated | unsaturated | unsaturated |
| Before | 0 | 3.5 +/− 0.5 | 18.9 | 14.8 | 66.3 | — | — | — |
| packing | | | | | | | | |
| 100% N2 | 2 wks | 3.8 +/− 0.6 | 19.82 | 15.92 | 64.15 | +0.92 | +1.12 | −2.15 |
| H2 + N2 | | 3.4 +/− 0.6 | 25.98 | 14.46 | 59.56 | +7.08 | −0.34 | −6.74 |
| 100% N2 | 4 wks | 3.5 +/− 0.6 | 18.7 | 15.16 | 66.14 | −0.2 | +0.36 | −0.16 |
| H2 + N2 | | 3.5 +/− 0.6 | 23.19 | 12.45 | 64.36 | +4.29 | −2.35 | −1.94 |

MG: fatty materials
AG: Fatty acids

The modified 4.5% H2/95.5% N2 atmosphere impacted the profile of the fatty acids in the leaven by increasing the composition in saturated fatty acids and proportionately reducing the composition in unsaturated fatty acids while packaging in 100% N2 completely stabilised the composition in fatty acids in the leaven.

Saturated fatty acids are lipids with no double link in their chemical structure. In foods, they are mainly found in products of animal origin (butter, meats, etc), while unsaturated fatty acids have one or more double links in their structure and are found mainly in oily fish and foods of vegetable origin, as is the case with wheat leaven in this example. Unsaturated fatty acids are to be preferred in foods as they are recognised for their beneficial effects on health, in particular for their contribution to healthy functioning of the cardiovascular system.

Packaging in 100% N2 acording to the invention, thus allows the nutritional properties of leaven to be well preserved, while an atmosphere containing hydrogen tends to encourage transformation into saturated fatty acids.

Unexpectedly, packaging in nitrogen not only preserves the intrinsic characteristics of leaven (colour, physico-chemical and nutritional composition), but also prolongs the life of its microflora, in particular preserving the longest living lactic bacteria, compared with classical packaging in air.

1. A product consisting of leaven or the like, living, having a percentage by weight of dry material of less than 90%, characterised in that the product is contained in an atmosphere of nitrogen with a purity of greater than 97% by volume in a package of a material with a high gas barrier and with a gas tight seal.

2. A product according to claim 1, characterised in that the packaging material has a permeability coefficient to O₂ of less than 10 cm³/m²/24 h.

3. A product according to claim 1, characterised in that the packaging material is a barrier to water vapour.

4. A product according to claim 1, characterised in that the material consists of one or more layers of PVDC, OPA, EVOH or aluminium or a mixture of these.

5. A product according to claim 1, characterised in that the material consists of glass, silica and/or aluminium.

6. A product according to claim 1, characterised in that the material is anti-UV.

7. A process of manufacturing the product claim 1, characterised in that the living leaven or the like, is placed in a container made of a gas barrier material in an atmosphere of more than 97% pure nitrogen by volume and the container is closed with a gas tight seal.

8. A process according to claim 7, characterised in that nitrogen with a purity of between 99 and 100% by volume is used.

9. A product according to claim 1, wherein the purity of nitrogen is between 99 and 100% by volume.

10. A method for keeping lactic bacteria in leaven in a cultivable state comprising packaging leaven in a package of a material with gas barrier filled with nitrogen with a purity greater than 97% by volume and tightly sealing the package.

11. The method of claim 10, wherein the purity of nitrogen is between 99% and 100% by volume.

12. The method of claim 10, wherein the material has a permeability coefficient to O₂ of less than 10 cm³/m²/24 h.

13. A method for maintaining the content of unsaturated fatty acids in leaven comprising packaging leaven in a package of a material with gas barrier filled with nitrogen with a purity greater than 97% by volume and tightly sealing the package.

14. The method of claim 13, wherein the purity of nitrogen is between 99% and 100% by volume.

15. The method of claim 13, wherein the material has a permeability coefficient to O₂ of less than 10 cm³/m²/24 h.
